(19) United States
(12) Patent Application Publication
(10) Pub. No.: US 2025/0082586 A1
(43) Pub. Date: Mar. 13, 2025

(54) COMPOSITIONS FOR TREATING INFLAMMATORY, NEUROLOGIC AND/OR VASCULAR CONDITIONS AND METHODS OF USE THEREOF

(71) Applicant: Brian Stuart Murphy, Newport Coast, CA (US)

(72) Inventor: Brian Stuart Murphy, Newport Coast, CA (US)

(21) Appl. No.: 18/561,386

(22) PCT Filed: May 18, 2022
(86) PCT No.: PCT/US2022/029772

§ 371 Date: Nov. 16, 2023

Related U.S. Application Data
(60) Provisional application No. 63/191,668, filed on May 21, 2021.

(51) Int. Cl. A61K 31/13; A61K 9/24; A61K 9/48; A61K 31/00

(52) U.S. Cl. CPC A61K 31/13; A61K 9/209; A61K 9/4858; A61K 9/4866; A61K 31/658

(57) ABSTRACT

Described are pharmaceutical compositions for treating an inflammatory condition, a neurologic condition and/or a vascular condition, the composition including i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator. Further described are methods of treating inflammatory, neurologic and/or vascular conditions.

### CROSS-REFERENCE TO RELATED APPLICATIONS

This National Stage Utility Patent Application claims the benefit of and priority to PCT Patent Application No. PCT/US2022/029772, filed on May 18, 2022, titled Compositions For Treating Inflammatory, Neurologic And/Or Vascular Conditions And Methods Of Use Thereof, the contents of which are expressly incorporated herein by this reference as though set forth in their entirety and to which priority is claimed. International Patent Application No. PCT/2022/029772 claims the benefit of and priority to U.S. Provisional Patent Application No. 63/191,668, filed on May 21, 2021, the contents of which are expressly incorporated herein by this reference, and to which priority is claimed.

### FIELD

One or more embodiments of the disclosure relate to the field of pharmaceutical compositions for treating a inflammatory, neurologic and/or vascular conditions. In some embodiments, the inflammatory condition may be a multisystem inflammatory condition and the neurologic and/or vascular condition may be associated with the inflammatory condition. The disclosure also relates to one or more embodiments of a method for treating an inflammatory, neurologic and/or vascular conditions.

### BACKGROUND

Inflammatory, neurologic and/or vascular conditions can be painful and debilitating, affecting an individual's health and well-being. A number of palliative treatment options are available that provide relief from associated symptoms. However, there continues to be a need for new options to manage such conditions.

Inflammatory, neurologic and/or vascular conditions may arise from a variety of circumstances. Insult to neurological tissue, whether central nervous system or peripheral nervous system, may be caused by blunt force trauma (e.g., a concussion), post-surgical sequelac, chemical insult (e.g., chemotherapy or chemical agent), extreme metabolic changes such as elevated blood glucose in diabetes, an autoimmune response triggered by internal or external stimuli resulting in an inflammatory cascade coupled to activation of NLRP3-inflammasome complex or may be physiologically mediated (e.g., an immune response to an infectious insult or an autoimmune response to various triggers). The internal or external stimuli that trigger an autoimmune response may be infection, tissue damage from any cause, implantation of a medical device, metabolic imbalance and/or toxin exposure. The inflammasome subsequently activates Caspase-1, which in turn activates pro-inflammatory cytokines interleukin 1-beta (IL-1β) and interleukin-18 (IL-18) leading to further cytokine activation, including but not limited to interleukin 10 (IL-10), interleukin 17 (IL-17), tissue necrosis factor (TNF), leading to tissue and organ compromise secondary to inflammation and pyroptosis. Of particular concern is inflammation in the endovascular space that may lead to vascular spasm, edema and potentially the formation of blood clots all of which can further compromise blood flow to tissues, especially narrow-bore vessels found in the brain, heart, kidneys, eye, and the pulmonary vasculature.

A viral infection such as severe acute respiratory syndrome coronavirus 2 (SARS-CoV-2), the pathogen responsible for the coronavirus disease 2019 (COVID-19) pandemic, may also trigger inflammatory, neurologic and/or vascular conditions. COVID-19 is recognized as a multi-organ disease with a broad spectrum of manifestations including inflammatory and neurologic conditions. In some subjects, the effects of acute COVID-19 can be persistent and prolonged (i.e., “long haulers”), a syndrome characterized by persistent symptoms and/or delayed or long-term complications beyond four (4) weeks from symptom onset.

There exists a need in the art for pharmaceutical compositions and methods for treating inflammatory, neurologic and/or vascular conditions as described herein, and corresponding pharmaceutical compositions that can inactivate the NLRP-3-inflammasome complex to suppress inflammation.

### BRIEF SUMMARY

According to one or more embodiments, disclosed herein is a method of treating at least one of an inflammatory condition or a neurologic condition comprising administering to a subject i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator.

In one or more further embodiments, disclosed herein is a method of treating post-encephalitis and/or post COVID-19 syndrome comprising administering to a subject i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator.

In yet one or more further embodiments, disclosed herein is a pharmaceutical composition comprising i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator (e.g., to mitigate conditions negatively impacting neurological transmission such as Parkinson's disease, epilepsy, Alzheimer's disease, multiple sclerosis, and Huntington's disease or inflammation caused by physical trauma such as that seen in traumatic brain injuries like concussions).

In yet one or more further embodiments, disclosed herein is a pharmaceutical composition comprising i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator (e.g., to mitigate conditions negatively impacting neurological transmission such as Parkinson's disease, epilepsy, Alzheimer's disease, multiple sclerosis, and Huntington's disease or inflammation caused by physical trauma such as that seen in traumatic tissue injuries caused by surgery (e.g. transplantation), or chemically, such as the use of cancer chemotherapeutic agents).

### DEFINITIONS

As used herein, the singular forms “a,” “an,” and “the” include plural references unless the context clearly indicates otherwise. Thus, for example, reference to “a cannabinoid” includes a single cannabinoid as well as a mixture of two or more different cannabinoid or a derivative, prodrug or analogue thereof; and reference to an “excipient” includes a single excipient as well as a mixture of two or more different excipients, and the like.

As used herein, the term “about” in connection with a measured quantity or time, refers to the normal variations in that measured quantity or time, as expected by one of ordinary skill in the art in making the measurement and exercising a level of care commensurate with the objective of measurement. In certain embodiments, the term “about” includes the recited number ±10%, such that “about 10” would include from 9 to 11, or “about 1 hour” would include from 54 minutes to 66 minutes.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

As used herein, the term “active agent” refers to any material that is intended to produce a therapeutic, prophylactic, or other intended effect, whether or not approved by a government agency for that purpose. This term with respect to a specific agent includes the pharmaceutically active agent, and all pharmaceutically acceptable salts, solvates and crystalline forms thereof, where the salts, solvates and crystalline forms are pharmaceutically active.

As used herein, the terms “therapeutically effective” and an “effective amount” refer to that amount of an active agent or the rate at which it is administered needed to produce a desired therapeutic result.

The term “subject” refers to a human or animal, who has demonstrated a clinical manifestation of an inflammatory, neurologic and/or vascular condition. The term “subject” may include a person or animal (e.g., a canine) who is a patient being appropriately treated by a medical caregiver for an inflammatory, neurologic and/or vascular condition.

The terms “treatment of” and “treating” include the administration of an active agent(s) with the intent to lessen the severity of a condition and/or a symptom.

The terms “prevention of” and “preventing” include the avoidance of the onset of a condition by a prophylactic administration of the active agent.

The term “condition” or “conditions” may refer to those conditions commonly recognized as inflammatory, neurologic and/or vascular disorders. “Conditions” may refer to neurologic injury or neurologic disease with acute or chronic sequelae that is physical and/or psychological in nature including, but not limited to, insult to neurological tissue (i.e., central nervous system or peripheral nervous system), for example, caused by blunt force trauma (e.g., a concussion), surgical trauma or manipulation of tissues such as tissue transplantation, chemical insult to neurological tissue (e.g., chemotherapy or exposure to a chemical agent), an autoimmune response triggered by internal or external stimuli resulting in an inflammatory cascade coupled to activation of NLRP3-inflammasome complex or may be physiologically mediated (e.g., an immune response to an infectious insult or an autoimmune response to various triggers), an encephalopathy, an acute pain condition, a chronic pain condition and/or a combination of any two or more of the foregoing, which can be treated, mitigated or prevented by a timely administration to a subject of an effective amount of a pharmaceutical composition as described herein. The condition may affect neurological tissue, the central nervous system, the peripheral nervous system, the brain, organs, ocular tissues, musculature, joints, the pulmonary system, hematologic system, cardiovascular system, neuropsychiatric system, renal system, endocrine system and/or gastrointestinal and hepatobiliary system. In certain embodiments, the term “condition” or “conditions” may refer to anxiety, post-traumatic stress syndrome (PTSD), depression, sleep disturbance, myalgic encephalomyelitis/chronic fatigue syndrome (MS/CFS), mononucleosis from Epstein-Barr virus or other herpes-related viruses, Lyme Disease, severe acute respiratory syndrome (SARS), COVID-19, post-COVID-19 syndrome, human immunodeficiency virus (HIV), hepatitis B (HBV), hepatitis C (HCV), herpesviridae, Ebola, fibromyalgia, autoimmune encephalitis, neuropathic pain, post-operative cognition deficit, Parkinson's disease, multiple sclerosis (MS), Alzheimer's disease, and/or any combination of two or more of the foregoing. In one or more embodiments, the term “condition” or “conditions” may additionally or alternatively refer to vasculitis, CNS vasculitis, vascular inflammation, vascular diseases involving activation of NLRP3, NLRP1, NLRC4 or AIM2 inflammasomes, vascular disease resulting from pathogen-driven stimuli, tissue injury or metabolic alterations in multiple cell types and different organs of the body, acute or chronic vascular injury, vasculitides diseases and disorders, Sjögren's disease, Kawasaki's disease, polyarteritis nodosa, Churg-Strauss angiitis, Wegner's granulomatosis, temporal arteritis or isolated angiitis of the central nervous system. The condition can also be caused by malaria or by a parasite such as trypanosomiasis or leishmaniasis

T_{1/2} refers to the time for the plasma concentration of an active agent to decrease by half.

The term “concurrently” as used herein means that a dose of one agent (e.g., a cannabinoid or a modulator) is administered prior to the end of the dosing interval of another agent (e.g., a cannabinoid or a modulator). For example, a dose of a cannabinoid with a particular dosing interval would be concurrently administered with a modulator dose when administered within the dosing interval of the cannabinoid.

The term “simultaneously” as used herein means that a dose of one agent is administered approximately at the same time as another agent, regardless of whether the agents are administered separately via the same or different routes of administration or in a single pharmaceutical composition or dosage form. For example, a dose of a cannabinoid may be administered separately from, but at the same time as, a dose of a modulator.

The term “sequentially” as used herein means that a dose of one agent is administered first and thereafter a dose of another agent is administered second. For example, a dose of a cannabinoid may be administered first, and thereafter a dose of a modulator may be administered second. The subsequent administration of the second agent may be inside or outside the dosing interval of the first agent.

The disclosed therapies can be prophylactic, i.e., to prevent or minimize the occurrence of a disease state or be therapeutic to actively treat the disease state.

### DETAILED DESCRIPTION

In one or more embodiments, described herein is a method of treating an inflammatory, neurologic and/or vascular condition comprising administering to a subject i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator. In some embodiments, the inflammation is NLRP3-inflammasome mediated inflammation associated with traumatic, chemical and/or physiologically-mediated neurologic injury. In at least one embodiment, disclosed herein is a method of treating post COVID-19 syndrome comprising administering to a subject i) a cannabinoid and ii) a modulator compound that acts as one or more of a glutamatergic NMDA receptor modulator, a 5-HT₃ receptor modulator, a nicotinic acetylcholine receptor modulator, a dopamine D2 receptor modulator, or a sigma-1 receptor modulator. In certain embodiments, the modulator compound is an NMDA receptor modulator agonist, antagonist, or mixed agonist-antagonist at one or more of the NMDA receptor modulator, the 5-HT₃ receptor modulator, the nicotinic acetylcholine receptor modulator, the dopamine D2 receptor modulator, or the sigma-1 receptor modulator. In particular embodiments, the modulator compound is memantine, a prodrug thereof, an analog thereof, a metabolite thereof, or a pharmaceutically acceptable salt thereof. In certain embodiments, the cannabinoid comprises one or more of a natural compound, a synthetic compound, or a semi-synthetic compound. In particular embodiments, the cannabinoid is natural, synthetic or semi-synthetic cannabidiol (CBD), CBD-hemisuccinate, a form of Δ8-tetrahydrocannabinol (Δ8-THC), Δ9-THC, and/or a pharmaceutically acceptable salt, prodrug, analog or metabolite thereof.

In some embodiments, the method includes administering cannabidiol or a pharmaceutically acceptable salt thereof and memantine or a pharmaceutically acceptable salt thereof. In certain embodiments, the cannabinoid and the modulator compound are administered to the subject via the same or different routes, for example, independently, by oral (e.g., an oral solid dosage form, a tablet, a capsule, a softgel, a powder, a suspension or a solution), sublingual, parenteral, ophthalmic (e.g., liquid drops, suspension drops, topical cream, topical ointment, topical lotion or topical gel), transdermal (e.g., subcutaneous by an implantable solid, semi-solid, gel or a viscous liquid), topical (e.g., a cream, an ointment, a lotion or a gel) and/or transgingival routes (e.g., a tape-like strip that is applied to the gum and dissolves allowing absorption of drug directly into the bloodstream and bypassing the gastrointestinal tract and first-pass metabolism in the liver).

In certain embodiments, the inflammatory, neurologic and/or vascular condition affects neurological tissue, the central nervous system, the peripheral nervous system, the ocular system, the brain, organs, musculature, joints, the pulmonary system, hematologic system, cardiovascular system, neuropsychiatric system, renal system, endocrine system and/or gastrointestinal and hepatobiliary system. In some embodiments, the inflammatory, neurologic and/or vascular condition is anxiety, post-traumatic stress syndrome (PTSD), depression, sleep disturbance, myalgic encephalomyelitis/chronic fatigue syndrome (MS/CFS), mononucleosis from Epstein-Barr virus, Lyme Disease, severe acute respiratory syndrome (SARS), COVID-19, post-COVID-19 syndrome, human immunodeficiency virus (HIV), hepatitis B (HBV), hepatitis C (HCV), herpesviridae, Ebola, fibromyalgia, autoimmune encephalitis, neuropathic pain, post-operative cognition deficit, Parkinson's disease, multiple sclerosis (MS), Huntington's disease, Alzheimer's disease, and/or any combination of two or more of the foregoing.

In certain embodiments, the methods treat one or more of anxiety, post-traumatic stress syndrome (PTSD), depression, sleep disturbance, myalgic encephalomyelitis/chronic fatigue syndrome (MS/CFS), mononucleosis from Epstein-Barr virus, Lyme Disease, severe acute respiratory syndrome (SARS), COVID-19, post-COVID-19 syndrome, human immunodeficiency virus (HIV), hepatitis B (HBV), hepatitis C (HCV), herpesviridae, Ebola, fibromyalgia, autoimmune encephalitis, neuropathic pain, post-operative cognition deficit, Parkinson's disease, multiple sclerosis (MS), Alzheimer's disease, and/or any combination of two or more of the foregoing. In certain embodiments, the methods include administering a cannabinoid, a modulator or both, each independently in the form of nanoparticles, a nanoemulsion or nanocrystals.

In one or more embodiments, described herein is a pharmaceutical composition comprising i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator. In certain embodiments, the NMDA receptor modulator is an agonist, an antagonist, or a mixed agonist-antagonist at one or more of the NMDA receptor modulator, the 5-HT₃ receptor modulator, the nicotinic acetylcholine receptor modulator, the dopamine D2 receptor modulator, or the sigma-1 receptor modulator. In particular embodiments, the modulator compound is memantine, a prodrug thereof, an analog thereof, a metabolite thereof or a pharmaceutically acceptable salt thereof. In certain embodiments, the cannabinoid comprises one or more of a natural compound, a synthetic compound, or a semi-synthetic compound. In particular embodiments, the cannabinoid is natural, synthetic or semi-synthetic cannabidiol (CBD), CBD-hemisuccinate, a form of Δ8-tetrahydrocannabinol (Δ8-THC), Δ9-THC, and/or a pharmaceutically acceptable salt, prodrug, analog or metabolite thereof.

In some embodiments, the pharmaceutical composition includes cannabidiol or a pharmaceutically acceptable salt thereof and memantine or a pharmaceutically acceptable salt thereof. In certain embodiments, the cannabinoid and the modulator compound are administered to the subject via the same or different routes, for example, independently by oral (e.g., an oral solid dosage form, a tablet, a capsule, a softgel, a powder, a suspension or a solution), parenteral (e.g., subcutaneous, intramuscular, intravenous, by needle, implant, infusion, a pump device, an external pump, a subcutaneous pump, a subcutaneous depot or an implantable pump, by an implantable solid, semi-solid, gel or viscous liquid), sublingual, rectal (e.g., by suppository), vaginal, ocular (e.g., liquid drops, suspension drops, topical cream, topical ointment, topical lotion or topical gel), nasal, dermal (e.g., intradermal, transdermal), inhalation, nebulization, topical routes (e.g., a cream, ointment, lotion, gel) or transgingival.

In certain embodiments, at least one pharmaceutical composition is in the form of nanounits including one or more of nanoparticles, nanospheres, nanocapsules or nanocrystals, or a nanoemulsion. In at least one embodiment, the oral solid dosage form is a tablet, capsule, gelcap, sublingual dosage form or depot delivery system comprising nanounits. In some embodiments, at least one agent is enmeshed in a physical blend of hylaronan and methyl-cellulose. The cannabinoid and the modulator compound may be in the same dosage form or in different dosage forms. In some embodiments, the method may include administering an adjunctive medicine to treat the inflammatory, neurologic and/or vascular condition. The adjunctive medicine can include at least one of n-acetyl cysteine (NAC), a topoisomerase, a non-steroidal anti-inflammatory drug (NSAID) or a steroid.

Without being bound by any particular theory, the connection of the memantine mechanism of action (e.g., NMDA receptor antagonism) and cannabinoids (e.g., the CB-1, CB-2, and TRPV receptors) is the inactivation of the NLRP-3/inflammasome complex. It is believed that treating an inflammatory, neurologic and/or vascular condition with a modulator compound and a cannabinoid administered together or separately can suppress inflammation.

### Active Agents

According to one or more embodiments, pharmaceutical compositions as described herein include a cannabinoid, a modulator compound, or both a cannabinoid and a modulator compound. According to one or more embodiments, a suitable cannabinoid compound for pharmaceutical compositions (and methods) as described herein includes, but is not limited to, a natural cannabinoid compound (e.g., a phytocannabinoid, an endocannabinoid, a metabolite), a synthetic cannabinoid compound (e.g., chemically synthesized), a semi-synthetic cannabinoid compound (e.g., a derivative of a natural cannabinoid) or a combination of any two or more of the foregoing. In some embodiments, the cannabinoid compound includes one or more of an isolate, a full-spectrum cannabinoid or a broad spectrum cannabinoid. A full-spectrum cannabinoid compound includes all biologically active constituents present in a cannabis plant, that is, the cannabinoid compound may include plant material and/or all compounds (e.g., all cannabinoids) of a cannabis plant. In one or more embodiments, the cannabinoid compound includes plant material from a Cannabis Sativa L. plant, a Cannabis Indica plant, a Cannabis Ruderalis plant, a Cannabis hybrid of any two of the foregoing, a derivative thereof, a prodrug thereof, an analog thereof, a metabolite thereof and/or a salt thereof. In embodiments, the cannabinoid constituent may also be synthetically produced via chemical production or a product of biosynthetic production using yeast, E.coli, algae, or other living forms.

In one or more embodiments, the cannabinoid may be an anti-inflammatory. Without being bound by any particular theory, it is believed that cannabinoids may be useful to help manage acute or chronic pain and inflammation (i.e., as an anti-inflammatory agent) associated with, for example, inflammatory, neurologic and/or vascular conditions. Agonist-activated cannabinoid receptors can modulate nociceptive thresholds, inhibit the release of pro-inflammatory molecules and display synergistic effects with other systems that influence analgesia, particularly the endogenous opioid system. As such, cannabinoids can act on inflammation through mechanisms different from those of agents such as nonsteroidal anti-inflammatory drugs (NSAIDs) and steroidal compounds. As a class, the cannabinoids also may be free from the adverse effects associated with NSAIDs and steroidal compounds. Their clinical development may provide a new approach to treatment of diseases characterized by acute and chronic inflammation and subsequent fibrosis.

In some embodiments, the cannabinoid may be an anti-fibrotic agent. Fibrosis in connective tissue develops as a reparative response to injury or damage and is often a sequelae of inflammation. Fibrosis may refer to connective tissue deposition that occurs as part of normal healing or to excess tissue deposition that occurs as a pathological process. Examples of fibrosis are intestinal fibrosis, skin scarring such as cicatrix and liver fibrosis. Without being bound by any particular theory, it is believed that certain cannabinoids, for example, cannabidiol (i.e., natural, synthetic or semi-synthetic), may reduce fibrosis in tissue and scarring (e.g., externally and/or internally). Some cannabinoids, such as tetrahydrocannabinol (THC), may increase fibrosis of tissue and scarring.

In one or more embodiments, the cannabinoid may be an anti-infective agent. Cannabinoids, such as Δ⁹-THC and CBD, have been found to have antimicrobial, bacteriostatic and bactericidal activity, for example, against Gram-positive pathogens (e.g., methicillin-resistant Staphylococcus aureus or MRSA isolates). In addition to THC and CBD, the cannabinoids cannabidiolic acid (CBDA), cannabigerol (CBG), cannabigerolic acid (CBGA) and cannabinol (CBN) have been found to have antibacterial activity against multidrug-resistant (MDR) S. aureus isolates.

In some embodiments, the cannabinoid may be a neuroprotective agent. The biological effects of cannabinoids are mediated by two cannabinoid receptors: CB1 and CB2. The CB1 receptor is the prominent type in the central nervous system (CNS) and may be therapeutic for neuropsychological disorders and neurodegenerative diseases. For example, overactivation of glutamate N-methyl-D-aspartate receptor (NMDAR) increases the cytosolic concentrations of calcium and zinc, which significantly contributes to neural death. Because cannabinoids prevent the NMDAR-mediated increase in cytosolic calcium, they also may control the rise of toxic free zinc ions, as well as the processes implicated in this phenomenon. It has been found that the CB1 receptor can regulate NMDAR function when the receptor is coupled to the histidine triad nucleotide-binding protein 1 (HINT1). In another example, THC may preserve retinal ganglion cells. The retina is anatomically and developmentally an extension of the CNS, and the retina and the brain are connected by the optic nerve, the axons of the ganglion cells, through the lateral geniculate nucleus. Cannabinoids have shown neuroprotective effects in different models of retinal neurodegeneration.

Suitable cannabinoid compounds for pharmaceutical compositions as described herein include, but are not limited to, cannabinol, cannabidiol, cannabidiol hemisuccinate, cannabidiol valine hemisuccinate, CBD-Di-Alaninate-Di-Hemisuccinate, CBD-Di-Valinate-Di-HS, CBD-Mono-Valinate-Mono-Hemisuccinate, CBD-monovalinate-dihemisuccinate, cannabigerol, cannabichromene, cannabidiolic acid, cannabigerolic acid, Δ⁹-tetrahydrocannabinol, Δ⁸-tetrahydrocannabinol, 11-hydroxy-tetrahydrocannabinol, 11-hydroxy-Δ⁹-tetrahydrocannabinol, Δ¹¹-tetrahydrocannabinol, tetrahydrocannabivarin, anandamide, 2-arachidonoylglyerol, dronabinol, levonantradol, nabilone, a derivative thereof, a prodrug thereof, an analog thereof, a metabolite thereof and/or a pharmaceutically acceptable salt thereof. In some embodiments, the pharmaceutical composition is free of tetrahydrocannabinol, a derivative thereof, a prodrug thereof, an analog thereof, a metabolite thereof and/or a pharmaceutically acceptable salt thereof. In one or more embodiments, the cannabinoid compound includes one or more of THC, CBD, CBDA, CBC, CBCA CBG, CBGA, CBN, the cannabinoid-varin class of compounds, a derivative thereof, a prodrug thereof, an analog thereof, a metabolite thereof and/or a pharmaceutically acceptable salt thereof.

Suitable natural cannabinoid compounds for pharmaceutical compositions (and methods) as described herein include, but are not limited to, the phytocannabinoids Δ⁹-tetrahydrocannabinol (Δ⁹-THC), Δ⁸-tetrahydrocannabinol (Δ⁸-THC), cannabinol (CBN), cannabidiol, cannabigerol, cannabichromene (CBC), cannabivarin (CBV), cannabidivarin (CBDV), tetrahydrocannabivarin (THCV), cannabinodiol (CBND), cannabielsion (CBE), cannabicyclol (CBL), cannabitriol (CBT) cannabinol, cannabigerol (CBG), cannabidiolic acid, cannabigerolic acid, the endocannabinoids anandamide (AEA) or 2-arachidonoylglyerol (2-AG), cannabinoid-varin compounds, the metabolites 11-hydroxy-tetrahydrocannabinol, 11-hydroxy-Δ⁹-tetrahydrocannabinol, Δ¹¹-tetrahydrocannabinol, tetrahydrocannabivarin, N-desmethylclobazam, hydroxylated 7-COOH derivatives of CBD, and/or any combination of two or more of the foregoing., In one or more embodiments, a natural cannabinoid compound suitable for pharmaceutical compositions described herein includes one or more of THC, CBD, CBDA, CBG, CBGA, CBN and/or a metabolite thereof.

Suitable synthetic cannabinoid compounds for pharmaceutical compositions (or methods) according to at least one embodiment herein include, but are not limited to, dronabinol (e.g., Marinol®, Syndros®), nabilone (e.g., Ceasamet®), plant-derived cannabidiol (e.g., Epidiolex®) a derivative thereof, a prodrug thereof, an analog thereof, a metabolite thereof, a pharmaceutically acceptable salt thereof and/or any combination thereof. In one or more embodiments, the cannabinoid compound is any cannabinoid made in a laboratory (e.g., through chemical synthesis).

Suitable semi-synthetic cannabinoid compounds for pharmaceutical compositions (or methods) described herein include, but are not limited to, a derivative of a natural cannabinoid) Δ⁹-tetrahydrocannabivarin (THCV), an analogue of a natural cannabinoid, a homologue of a natural cannabinoid, a hydrogenated cannabinoid, cannabidiol-dimethylheptyl, a metabolite of a natural or synthetic cannabinoid, a derivative thereof, a prodrug thereof, an analog thereof, a metabolite thereof, a pharmaceutically acceptable salt thereof and/or any combination thereof.

The modulator compound of the pharmaceutical composition (and methods) as described herein may include one or more of an NMDA receptor modulator, a 5-HT₃ receptor modulator, a nicotinic acetylcholine receptor modulator, a dopamine D2 receptor modulator, a sigma-1 receptor modulator and/or a combination thereof. In at least one embodiment, the NMDA receptor modulator is an agonist, an antagonist, or a mixed agonist-antagonist at one or more of the NMDA receptor modulator, the 5-hydroxytryptamine (5-HT₃) receptor modulator, the nicotinic acetylcholine receptor modulator, the dopamine D2 receptor modulator, and/or the sigma-1 receptor modulator.

NMDA receptor modulators (e.g., glutamate modulators) mediate a slow component of excitatory synaptic transmission, and NMDA receptor dysfunction has been implicated in numerous neurological disorders. For example, NMDA receptors located in the retinal ganglion cells play a role in visual processing including contrast coding. The NMDA receptor modulates synaptic plasticity in the visual cortex, a process that contributes to visual function. According to at least one embodiment, the modulator compound is a NMDA receptor antagonist. Non-limiting examples of NMDA receptor antagonists include a glutamatergic NMDA receptor antagonist, memantine and/or combinations thereof.

In some embodiments, the modulator compound is memantine, a prodrug thereof, an analog thereof, a metabolite thereof or a pharmaceutically acceptable salt thereof. Memantine hydrochloride is approved for treating moderate to severe dementia in adults with Alzheimer's disease. It is believed that memantine treatment, when started in the early phase of the glaucomatous process, may help preserve the retinal ultrastructure and thus prevent neuronal injury in glaucoma. Glutamate-induced excitotoxicity is implicated in glaucoma and NMDA receptor antagonism, such as memantine, has been advocated as a potential strategy for retinal ganglion cell (RGC) preservation. Without being bound by any particular theory, it is believed that co-administration of a pharmaceutical composition containing both a cannabinoid (e.g., CBD) and memantine may be useful to treat inflammatory, neurologic and/or vascular conditions. For example, the co-administration of a cannabinoid and memantine may act synergistically to produce a therapeutic effect greater than the additive effect of each component alone. In embodiments, the co-administration of a pharmaceutical composition containing a cannabinoid (e.g., CBD) and memantine may act synergistically to reduce inflammation associated with inflammatory, neurologic and/or vascular conditions.

Serotonin [5-HydroxyTryptamine (5-HT)] is a neurotransmitter with seven families (5-HT1-5-HT7) and approximately 15 receptor subtypes. In at least one embodiment, the modulator is a 5-HT receptor agonist, antagonist or mixed agonist-antagonist. Serotonin modulates nociceptive response through 5-HT1, 5-HT2, 5-HT3, and 5-HT7 receptors in the central nervous system (CNS). 5-HT receptor modulators may be useful in the treatment of inflammatory, neurologic and/or vascular conditions. For example, 5-HT, a proinflammatory neurotransmitter, can activate 5-HT₃ receptors to depolarize vagal afferent neurons, whereas 5-HT₁ and 5-HT₂ receptor subtypes bear highest affinity for the endogenous ligand and are thought to exert an overall antinociceptive action, and models of persistent pain have suggested a role for 5-HT₃ receptor activation in the maintenance of pain. In some instances, 5-HT_{2A} antagonists and/or 5-HT₃ agonists may be effective as peripherally acting analgesic agents and/or analgesic adjuncts. These analgesic actions may be specific to situations in which 5-HT release contributes to the generation of pain. According to at least one embodiment, the modulator is a 5-HT₃ receptor antagonist. Non-limiting examples of 5-HT₃ receptor antagonists as a modulator for pharmaceutical compositions according to embodiments herein include dolasetron, granisetron, ondansetron, palonosetron or any combination of two or more of the foregoing.

In at least one embodiment, the modulator is a nicotinic acetylcholine receptor agonist, antagonist or mixed agonist-antagonist. Acetylcholine (ACh) activates both nicotinic and muscarinic acetylcholine receptors (AChRs). One class includes heteromeric nicotinic AChR subtypes comprised of the α2-α6 and β2-β4 units with high agonist affinity but insensitivity to snake toxin α-bungarotoxin (αBgt); a second class includes homomeric (i.e., α7, α8, or α9) or heteromeric pentamers (i.e., combined α7, α8, α9, or α10 subunits) with lower agonist affinity but with high sensitivity to aBgt. Without being bound by any particular theory, it is believed that the α7 agonist may have a neuroprotective effect against neurodegenerative disease (e.g., amyotrophic lateral sclerosis or ALS, Alzheimer's, Parkinson's, etc.). In some embodiments, the modulator may include a nicotinic acetylcholine receptor antagonist. Suitable nicotinic acetylcholine receptor antagonists include, but are not limited to, chlorisondamine, mecamylamine and/or combinations thereof.

In some embodiments, the modulator is a dopamine D2 receptor agonist, antagonist or mixed agonist-antagonist. Dopamine receptors play important roles in the activity dependent synaptic plasticity in CNS and multiple subtypes of dopamine receptors are expressed by retinal neurons. D1 dopamine receptors have been shown to regulate the developmental enhancement of ERG b-wave and the light response gain between bipolar and ganglion/amacrine cells. It has been found that deletion of the dopamine D2 receptor has opposite effect on the inner retinal light response in comparison with D1 dopamine receptor mutation, it preferentially regulates the retinal light responses after eye opening, and effects induced by mutation of the D2 dopamine receptor on ERG are light-sensitive. In at least one embodiment, the modulator compound may be a dopamine D2 receptor agonist. Suitable dopamine D2 receptor agonists include, but are not limited to, dopamine, ibopamine (dopamine analog), fenoldopam, bromocriptine (dopaminergic agonist with higher affinity for D2 than for D1-receptors) and/or combinations thereof.

In some embodiments, the modulator is a sigma-1 receptor agonist, antagonist or mixed agonist-antagonist. Retinal degenerative diseases are a major cause of untreatable blindness. A target for treatment of retinal disease is the transmembrane protein Sigma 1 Receptor (Sig1R). Without being bound by any particular theory, it is believed that Sig1R plays a key role in modulating retinal cellular stress and that it holds great promise as a target in retinal neurodegenerative disease. According to at least one embodiment, the modulator of pharmaceutical compositions (and methods) described herein includes a sigma-1 receptor agonist. Suitable sigma-1 receptor agonists include, but are not limited to, (+)-pentazocine, (+)-SKF10,047, 2-morpholin-4-ylethyl 1-phenylcyclohexane-1-carboxylate and 1-[2-(3,4-dimethoxyphenyl) ethyl]-4-(3-phenylpropyl)piperazine) and/or a combination of any two or more of the foregoing.

In one or more embodiments, the pharmaceutical composition includes cannabidiol, cannabidiol hemisuccinate, cannabidiol valine hemisuccinate, CBD-Di-Alaninate-Di-Hemisuccinate, CBD-Di-Valinate-Di-HS, CBD-Mono-Valinate-Mono-Hemisuccinate, CBD-monovalinate-dihemisuccinate, a derivative thereof, a prodrug thereof, an analog thereof, a metabolite thereof, a pharmaceutically acceptable salt thereof and/or any combination thereof; and memantine, a derivative thereof, a prodrug thereof, an analog thereof, a metabolite thereof, a pharmaceutically acceptable salt thereof and/or any combination thereof.

In some embodiments, the pharmaceutical composition per unit dose comprises a cannabinoid as described in one or more embodiments herein in an amount of about 2 mg to about 50 mg, about 2 mg to about 40 mg, about 5 mg to about 30 mg, about 2 mg to about 25 mg, about 10 mg to about 20 mg, about 5 mg to about 20 mg, about 10 mg to about 20 mg, about 15 mg to about 25 mg, or about 15 mg to about 20 mg or any individual amount or sub-range within these ranges. According to one or more embodiments, the pharmaceutical composition comprises the cannabinoid in an amount of about 0.1 percent by weight (wt %) to about 20 wt %, about 0.5 wt % to about 15 wt %, about 1 wt % to about 10 wt %, about 2 wt % to about 5 wt %, about 0.1 wt % to about 10 wt %, about 0.5 wt % to about 10 wt %, or any individual wt % or sub-range within these ranges based on the total weight of the composition.

In one or more embodiments, the pharmaceutical composition per unit dose comprises a modulator as described in one or more embodiments herein in an amount of about 2 mg to about 50 mg, about 2 mg to about 40 mg, about 5 mg to about 30 mg, about 2 mg to about 25 mg, about 10 mg to about 20 mg, about 5 mg to about 20 mg, about 10 mg to about 20 mg, about 15 mg to about 25 mg, or about 15 mg to about 20 mg or any individual amount or sub-range within these ranges. According to one or more embodiments, the pharmaceutical composition comprises the modulator in an amount of about 0.1 wt % to about 20 wt %, about 0.5 wt % to about 15 wt %, about 1 wt % to about 10 wt %, about 2 wt % to about 5 wt %, about 0.1 wt % to about 10 wt %, about 0.5 wt % to about 10 wt %, or any individual wt % or sub-range within these ranges based on the total weight of the composition.

In at least one embodiment, the pharmaceutical composition per unit dose includes a cannabinoid (e.g., CBD) and a modulator (e.g., memantine) as described in one or more embodiments herein each individually present in an amount of about 2 mg to about 50 mg. about 2 mg to about 40 mg, about 5 mg to about 30 mg, about 2 mg to about 25 mg, about 10 mg to about 20 mg, about 5 mg to about 20 mg, about 10 mg to about 20 mg, about 15 mg to about 25 mg, or about 15 mg to about 20 mg or any individual amount or sub-range within these ranges. According to one or more embodiments, the pharmaceutical composition includes a weight ratio of the cannabinoid (e.g., CBD) to the modulator (e.g., memantine) of about 1:100 to about 100:1, about 1:50 to about 50:1, about 1:25 to about 25:1, about 1:20 to about 20:1, about 1:15 to about 15:1, about 1:10 to about 10:1, about 1:5 to about 5:1, about 1:2 to about 2:1 or any individual weight ratio or sub-range within these ranges.

In some embodiments, the pharmaceutical composition according to embodiments herein includes a cannabinoid (e.g., CBD) and a modulator (e.g., memantine) each individually present in an amount of about 0.1 wt % to about 20 wt %, about 0.5 wt % to about 15 wt %, about 1 wt % to about 10 wt %, about 2 wt % to about 5 wt %, about 0.1 wt % to about 10 wt %, about 0.5 wt % to about 10 wt %, or any individual wt % or sub-range within these ranges based on the total weight of the composition. According to one or more embodiments, the pharmaceutical composition includes a weight ratio of the cannabinoid (e.g., CBD) to the modulator (e.g., memantine) of about 1:100 to about 100:1, about 1:50 to about 50:1, about 1:25 to about 25:1, about 1:20 to about 20:1, about 1:15 to about 15:1, about 1:10 to about 10:1, about 1:5 to about 5:1, about 1:2 to about 2:1 or any individual weight ratio or sub-range within these ranges.

In one or more embodiments, pharmaceutical compositions described herein can include a supplement, a derivative thereof, a prodrug thereof, an analog thereof, a metabolite thereof and/or a salt thereof. Suitable supplements include, but are not limited to, an amino acid, a vitamin, a mineral, an herb and/or any combination thereof. Suitable amino acids include, but are not limited to, n-acetyl cysteine (NAC), cysteine, glutathione, glycine, L-alanine, β-alanine, α-aminoadipic acid, α-aminobutyric acid, γ-aminobutyric acid, α-aminoisobutyric acid, arginine, asparagine, aspartic acid, citrulline, creatine, glutamic acid, histidine, cystine, leucine, lysine, norleucine, ornithine, phenylalanine, phosphoserine, sarcosine, threonine, valine, L-theanine and/or any combination of two or more of the foregoing. According to some embodiments, pharmaceutical formulations include a cannabinoid (e.g., CBD), a modulator (e.g., memantine) and an amino acid (e.g., NAC). NAC is an acetylated derivative of the natural amino acid, L-cysteine and possesses mucolytic, anti-collagenolytic and antioxidant properties. NAC modulates the cellular redox status to influence inflammatory pathways, leading to decreased nuclear factor-kappa B activity, which regulates several proinflammatory genes that regulate the inflammation pathways. One or more pharmaceutical compositions according to embodiments herein can include a cannabinoid (e.g., CBD), a modulator (e.g., memantine) and NAC. Without being bound by any particular theory, the NAC may provide an additive or synergistic effect in treating inflammatory, neurologic and/or vascular conditions.

The amino acid may be present in the pharmaceutical formulation per unit dose in an amount of about 1 wt % to about 10 wt %, about 2 wt % to about 7 wt %, about 4 wt % to about 5 wt %, or any individual wt % or sub-range within these ranges.

Suitable vitamins for pharmaceutical formulations according to embodiments herein include, but are not limited to, one or more of a retinol ester, a retinal ester or a retinyl ester (collectively vitamin A), beta-carotene, thiamine (vitamin B1), L-ascorbic acid (vitamin C), one or more of α-tocopherol, β-tocopherol, γ-tocopherol, Δ-tocopherol, α-tocotrienol, β-tocotrienol, γ-tocotrienol or Δ-tocotrienol (collectively vitamin E), copper (e.g., cupric oxide), zinc (e.g., zinc oxide), lutein, zeaxanthin, omega-3 fatty acids and/or combinations of any two or more of the foregoing. Each vitamin individually may be present in the pharmaceutical formulation per unit dose in an amount of about 1 mg to about 1,000 mg, about 1 IU to about 1,000 IU, or any individual amount or sub-range within these ranges.

Suitable minerals for pharmaceutical compositions according to embodiments herein include, but are not limited to, calcium, phosphorus, potassium, sodium, chloride, magnesium, iron, zinc, iodine, sulfur, cobalt, copper, silver, fluoride, manganese, selenium and/or a combination of any two or more of the foregoing.

Suitable herbs for pharmaceutical compositions according to embodiments herein include, but are not limited to, phytonutrients, baicalin, cerium trichloride, cerium oxide, coenzyme Q10, curcumin, epigallocatechin gallate (EGCG), green tea extract, resveratrol, ursolic acid, a fruit extract, a vegetable extract, an herb extract, curry murraya koenigil leaf, psidium guajava, sesania grandiflora, emblic, sea kelp, mushroom, lemon peel, holy basil leaf, annatto, moringa leaf, apple, beet root, broccoli, carrot, spinach, tomato, strawberry, cherry, blackberry, green bell pepper, brussels sprout, ginger, blueberry, garlic, green onion, raspberry, parsley, cauliflower, red cabbage, asparagus, celery, cucumber, kale, peppermint leaf, or any combination of two or more of the foregoing. According to embodiments, the herbs may be present in the pharmaceutical formulation in an amount of about 10 mg to about 2000 mg, or any individual amount or sub-range within this range.

### Pharmaceutically Acceptable Excipients

Pharmaceutical compositions according to one or more embodiments herein can further include one or more pharmaceutically acceptable excipients. Examples of possible pharmaceutically acceptable excipients are described in the Handbook of Pharmaceutical Excipients, American Pharmaceutical Association (2012), which is incorporated by reference herein. Suitable excipients for pharmaceutical compositions according to various embodiments herein include, but are not limited to, plasticizers, colorants, lubricants, thermal lubricants, antioxidants, buffering agents, disintegrants or granulating agents, binding agents, diluents, glidants, anti-adherants, sweeteners, chelating agents, granulating agents, bulking agents, flavorants, surfactants, solubilizers, stabilizers, hydrophilic polymers, hydrophobic polymers, waxes, lipophilic materials, absorption enhancers, preservative, absorbent, cross-linking agents, bioadhesive polymers, pore formers, osmotic agents, polycarboxylic acids, and combinations of any two or more of the foregoing.

Examples of suitable binding agents include, but are not limited to, cellulosic polymers (e.g., hydroxypropylmethylcellulose, hydroxypropylcellulose, hydroxyethylcellulose, etc.), polyethylene glycol, an acrylic polymer, an acrylic copolymer, a graft copolymer of polyvinyl alcohol and polyethylene glycol, a polyvinyl alcohol, alginic acid, sodium alginate, starch, pregelatinized starch, sucrose, guar gum, salts thereof, derivatives thereof and combinations thereof. Additional binders include, but are not limited to, natural or synthetic waxes, fatty alcohols (e.g., lauryl, myristyl, stearyl, cetyl or cetostearyl alcohol), fatty acids, including, but not limited to, fatty acid esters, fatty acid glycerides (e.g., mono-, di-, and tri-glycerides), hydrogenated fats, hydrocarbons, stearic acid, hydrophobic and hydrophilic materials having hydrocarbon backbones, acacia, tragacanth, sucrose, gelatin, glucose, cellulose materials (e.g., methylcellulose and sodium carboxymethylcellulose (e.g., Tylose™)), magnesium aluminum silicate, polysaccharide acids, bentonites, polyvinylpyrrolidone (povidone), polymethacrylates, and pregelatinized starch (such as National™ 1511 and Starch 1500). Suitable waxes include, for example, beeswax, glycowax, castor wax, carnauba wax and other wax-like substances. A “wax-like” substance is defined as any material which is normally solid at room temperature and has a melting point of from about 30° C. to about 100° C.

Additional examples of binders which may be used include, but are not limited to, digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils, natural and synthetic waxes and polyalkylene glycols. In certain embodiments, hydrocarbons having a melting point of between 25° C. and 90° C. may be included. Of the long-chain hydrocarbon binder materials, fatty (aliphatic) alcohols can be incorporated into the mixture according to certain embodiments.

Examples of suitable disintegrants include, but are not limited to, sodium starch glycolate, clays (such as Veegum™ HV), celluloses (such as purified cellulose, methylcellulose, sodium carboxymethylcellulose, and carboxymethylcellulose), cross-linked sodium carboxymethylcellulose, starch, cross-linked polyvinylpyrrolidone (e.g., crospovidone), alginates, cornstarches and pre-gelatinized corn starches (such as National™ 1551 and National™ 1550), gums (such as agar, guar, locust bean, pectin, and tragacanth) and mixtures thereof. Disintegrants can be added at any suitable step during the preparation of the pharmaceutical compositions, such as prior to granulation or during a lubrication step prior to compression or encapsulation.

Suitable bulking agents include, but are not limited to, starches (e.g., corn starch), microcrystalline cellulose, lactose (e.g., lactose monohydrate), sucrose, dextrose, mannitol, calcium phosphate and dicalcium phosphate.

According to certain embodiments, the pharmaceutical compositions may include a plasticizer. Plasticizers may interact with hydrophobic materials resulting in a lower viscosity of the mixture as compared to the mixture without the plasticizer when measured under the same conditions. Certain plasticizers may lower the glass transition temperature (Tg) of hydrophobic materials. Suitable plasticizers include, but are not limited to, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers may include ethylene glycol, 1,2-butylene glycol, 2,3-butylene glycol, styrene glycol, diethylene glycol, tricthylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, tricthyl citrate, acetyl tricthyl citrate, tributyl citrate and allyl glycolate.

In at least one embodiment, the pharmaceutical composition includes a glidant. A glidant is an excipient that improves the flow characteristics of a compressible powder such as tablet ingredients or granules. Suitable glidants include, but are not limited to, silicon dioxide, colloidal silicon dioxide and the like.

Suitable diluents useful in pharmaceutical compositions as described herein include, but are not limited to, lactose (e.g., lactose (anhydrous), lactose (spray dried), lactose monohydrate), starch (e.g., directly compressible starch), mannitol, sorbitol, dextrose monohydrate, microcrystalline cellulose, dibasic calcium phosphate dihydrate, sucrose-based diluents, confectioner's sugar, monobasic calcium sulfate monohydrate, calcium sulfate dihydrate, calcium lactate trihydrate granular, dextrates (e.g., Emdex™), dextrose (e.g., Cerelose™), inositol, hydrolyzed cereal solids such as the Maltrons™ and Mor-Rex™, amylose, powdered cellulose (e.g., Elcema™), calcium carbonate, glycine, bentonite, polyvinylpyrrolidone, and the like.

Suitable lubricants include, but are not limited to, glyceryl behenate (Compritol™ 888), metallic stearates (e.g., magnesium, calcium and sodium stearates), stearic acid, hydrogenated vegetable oils (e.g., Sterotex™), talc, waxes such as beeswax and carnauba wax, silica, fumed silica, colloidal silica, calcium stearate, long chain fatty alcohols, boric acid, sodium benzoate and sodium acetate, sodium chloride, DL-Leucine, polyethylene glycols (e.g., Carbowax™ 4000 and Carbowax™ 6000), sodium oleate, sodium benzoate, sodium acetate, sodium lauryl sulfate, sodium stearyl fumarate (Pruv™), magnesium lauryl sulfate, stearic acid, stearyl alcohol, mineral oil, paraffin, micro crystalline cellulose, glycerin, propylene glycol and combinations thereof.

Suitable anti-adherents include, but are not limited to, talc, cornstarch, colloidal silicone dioxide (Cab-O-Sil™), DL-Leucine, sodium lauryl sulfate, and metallic stearates.

Other excipients (such as colorants, flavorants and sweeteners) can be utilized in embodiments of the pharmaceutical compositions where they impart little to no deleterious effect on the stability of the pharmaceutical composition.

In certain embodiments, the pharmaceutical composition may include a film coat. The film coat may include, but is not limited to, hydroxypropylmethylcellulose, polyethylene glycol, polyvinyl alcohol or a mixture of any two or more thereof.

### Dosage Forms

According to some embodiments, pharmaceutical compositions as described herein are formulated for oral administration. Suitable dosage forms for oral administration include, but are not limited to, oral solid dosage form, a tablet, a capsule, a softgel, a powder, a suspension, an oral solution, a tape-like strip that is applied to the gum and dissolves allowing absorption of drug directly into the bloodstream and bypassing the gastrointestinal tract and first-pass metabolism in the liver, nanoparticles, nanocrystals, nanoemulsions and/or any combination of two or more of the foregoing. Example oral dosage forms can include a cannabinoid (e.g., CBD) and/or a modulator (e.g., memantine) and one or more excipient chosen from an antioxidant, a pH adjusting component (e.g., hydrochloric acid, sodium hydroxide), a cosolvent, an emulsifier, sodium alginate, a penetration enhancer, a preservative, an emollient, polyethylene glycol, polyvinyl alcohol, propylene glycol, povidone, water, saline, normal saline, glycerin, mineral oil, sodium borate, boric acid, sodium chloride, calcium chloride, magnesium chloride, potassium chloride, potassium sorbate, sodium bicaronatemannitol, sodium phosphate, carboxymethylcellulose, hyaluronan, hyaluronic acid, dextrose, dextran, glycerin, hypromellose, hydroxyethylcellulose, hydroxypropylmethylcellulose, a polysaccharide, β-d-mannuronic acid and α-1 glucuronic acid residues, gellan gum, glycerin, mineral oil, castor oil, ethylenediaminetetraacetic acid (EDTA), poloxamer, white petrolatum, benzalkonium chloride, edetate disodium, anhydrous liquid lanolin, erythritol, levocarnitine, seed oil, and/or combinations of any two or more of the foregoing. The cannabinoid and/or modulator may be encapsulated within a hard or soft shell capsule. In some embodiments, the cannabinoid and/or modulator may be suspended in a liquid (e.g., an oil, a solvent, an antioxidant). In certain embodiments, the cannabinoid and/or modulator may be in the form of granules, particles, powder, extrudates, nanounits, nanoparticles, nanocrystals and/or a nanoemulsion.

Pharmaceutical compositions described herein may be formulated for immediate release, controlled release or modified release (e.g., containing both an immediate release component and a controlled release component). In one or more embodiments, the cannabinoid and/or modulator can be layered over a core tablet, a core particle, and/or a capsule. In some embodiments, the pharmaceutical composition is an oral dosage form and contains an inert film coating.

In some embodiments, the pharmaceutical composition may be in the form of a sublingual film. The sublingual film may be formulated for immediate release, controlled release or modified release (e.g., containing both an immediate release component and a controlled release component). In one or more embodiments, the drug within the film andor coated over the film. The film may be biodegradable or non-biodegradable, bioresorbable or non-bioresorbable, bioerodible or non-bioerodible.

Mucoadhesive polymers for use in sublingual films can improve drug retention by mucoadhesion of a polymer excipient that attaches to, for example, the gums. The polymer-mucin bond may be used to entrap soluble, colloidal and/or particulate material containing the pharmaceutical composition on the gum surface. As such, these polymers can increase the pre-gastrointestinal residence time of a cannabinoid and/or a modulator at the gingival site. Viscosity-enhancing agents and mucoadhesive polymers may be used to increase the bioavailability. Suitable mucoadhesive polymers include, but are not limited to, a natural polymer, a synthetic polymer, chitosan, alginate, gellan gum, guar gum, carbomer, Eudragit and/or any combination of two or more of the foregoing. Alginate is a suitable anionic polymer that acts as a penetration enhancer and has a high mucoadhesive strength. A pharmaceutical composition according to embodiments herein may be impregnated in, coated on and/or a applied on the mucoadhesive polymer.

The cannabinoid (e.g., CBD), modulator component (e.g., memantine), excipients and/or supplements according to embodiments herein may be in the form of nanoparticles. nanocrystals, nanoemulsions (e.g., a suspension of nanoparticles or nanocrystals in a fluid), nanocapsules, nanospheres and/or combinations thereof. Polymeric NPs have shown great potential for targeted delivery of drugs for the treatment of several diseases. Nanoparticles, nanocrystals, nanocapsules and nanospheres for use in pharmaceutical compositions according to embodiments herein may have a particle size distribution of less than about 1,000 nm, less than about 500 nm, less than about 250 nm, less than about 200 nm or any individual size or sub-range within these ranges as measured by dynamic light scattering and 1H-NMR. In some embodiments, the nanoparticles or nanocrystals have a mean size of about 1 nm to about 1,000 nm or any individual size or sub-range within this range. In some embodiments, pharmaceutical compositions according to embodiments herein may include memantine loaded polylactide-co-glycolide (PLGA)—polyethylene glycol (PEG) nanoparticles, which may be efficacious in the treatment of inflammatory, neurologic and/or vascular conditions. The drug loading and/or incorporation of the memantine loaded PLGA-PEG nanoparticles may be about 1 mg/mL to about 20 mg/mL, about 4 mg/mL to about 10 mg/mL, or any individual concentration or sub-range within these ranges.

In some embodiments, the pharmaceutical compositions disclosed herein are in solid oral dosage form such as a pharmaceutically acceptable tablet or capsule. According to embodiments, mixtures or blends of dried particles, nanoparticles, nanocrystals and/or granules contain, for example, a cannabinoid (e.g., CBD) and/or a modulator (e.g., memantine), optionally a supplement and optionally one or more excipients compressed into tablets or encapsulated in pharmaceutically acceptable capsules. In some embodiments, the mixtures or blends of dried particles or granules may include a coating of a cannabinoid and/or a modulator, a substrate, which may or may not comprise the same or a different active agent as the coating, and optionally one or more excipients, which may then be compressed into tablets or encapsulated in pharmaceutically acceptable capsules. In some embodiments, a pharmaceutical composition according to embodiments herein may be in the form of a nanoemulsion including at least one cannabinoid (e.g., CBD) and/or at least one modulator (e.g., memantine), optionally one or more excipient and optionally one or more supplement. An example nanoemulsion of a pharmaceutical composition according to embodiments herein may include a cannabinoid, a modulator, an excipient, a supplement, canola oil, soyabean oil, liquid paraffin, oleic acid, tween 20, tween 80, span 20, span 80, cetyl pyridinium chloride (CPC), glycerol, ethylene glycol, di-ethylene glycol monohexine ether, propylene glycol, bromophenol blue dye, tryptan blue, 3-(4,5-dimethylthiazol-2-yl)-2,5-diphenyl tetrazolium bromide (MTT), 1,1-diphenyl-2-picrylhydrazyl (DPPH), trypsin ethylenediaminetetraacetic acid (EDTA) and/or any combination of two or more of the foregoing.

In certain embodiments, the drug combination of the present invention can be administered as a controlled release depot or implant. The depot or implant can be administered, e.g., subcutaneously. One or more of the agents can be in nanoparticle form. The slow release can be obtained by the use of a polymer such as polylactic glycolic acid.

### Methods of Preparation

Further described herein are methods of preparing pharmaceutical compositions for treating inflammatory, neurologic and/or vascular conditions according to various embodiments. Pharmaceutical formulations can be sterile, liquid, semi-solid, or solid and may contain one or more active pharmaceutical ingredient(s) (e.g., a cannabinoid, a modulator and/or a supplement) intended for ingestion, injection, absorption or any other route of administration as described herein. The choice of diluent and excipients used for the preparation of pharmaceutical compositions should not adversely affect either the stability of the final product or the bioavailability of the active ingredients at the site of action. In some embodiments, the pharmaceutical compositions include an antioxidant (e.g., vitamin E oil) to prevent degradation of the cannabinoid.

Nanoparticles, nanocrystals, nanocapsules and/or nanospheres (collectively referred to as nanounits) suitable for pharmaceutical compositions according to one or more embodiments herein, may be formed using solvent evaporation. Solvent evaporation may be used to prepare polymeric nanounits from a preformed polymer. Optionally, an oil-in-water (o/w) emulsion may be prepared, for example, in the preparation of nanospheres. The method can include preparing an organic phase comprising a polar organic solvent (e.g., ethyl acetate, dichloromethane, chloroform) and dissolving a polymer in the polar organic solvent to form a polymer solvent. Additionally, an active ingredient (e.g., a cannabinoid and/or a modulator) may be dissolved or dispersed within the polymer solvent to form the organic phase. In some embodiments, the method can include preparing an aqueous phase containing a surfactant (e.g., polyvinyl acetate, polyvinyl alcohol) and emulsifying the organic solution in the aqueous phase. The emulsified organic solution may then be processed using high-speed homogenization or ultrasonication, yielding a dispersion of nanodroplets. A suspension of nanounits may be formed by evaporating the polymer solvent, which may be allowed to diffuse through the continuous phase of the emulsion. The polymer solvent may be evaporated by, for example, continuous magnetic stirring at room temperature (in case of more polar solvents) or in a slow process of reduced pressure (as happens when using e.g., dichloromethane and chloroform). After the solvent has evaporated, the solidified nanounits can be washed and collected by centrifugation, followed by freeze-drying for long-term storage.

An emulsification/solvent diffusion method also may be used to prepare polymeric nanounits. The method can include the formation of an o/w emulsion between a partially water-miscible solvent containing a polymer and an active agent (e.g., a cannabinoid and/or a modulator), and an aqueous solution with a surfactant. The internal phase of this emulsion can include a partially hydro-miscible organic solvent, such as benzyl alcohol or ethyl acetate, which may be previously saturated with water in order to ensure an initial thermodynamic balance of both phases at room temperature. Subsequent dilution with a large amount of water can induce solvent diffusion from the dispersed droplets into the external phase, resulting in the formation of colloidal particles. Generally, this method may be used to produce nanospheres, but nanocapsules can also be obtained if a small amount of oil (such as triglycerides: C₆, C₈, C₁₀, C₁₂) is added to the organic phase. Depending on the boiling point of the organic solvent, this latter stage can be eliminated by evaporation or by filtration. The method can form nanounits with dimensions ranging from about 80 nm to 900 nm. This method can be applied to produce polymeric nanounits, despite the requirement of a high volume of the aqueous phase, which must be eliminated from the colloidal dispersion, and despite the risk of diffusion of a hydrophilic drug into the aqueous phase.

An emulsification/reverse salting-out method also may be used to prepare nanounits. The above described emulsification/solvent diffusion method can be considered a modification of the emulsification/reverse salting-out method. According to embodiments, the salting-out method is based on the separation of a hydro-miscible solvent from an aqueous solution, through a salting-out effect that can result in the formations of nanospheres. The main difference is the composition of the o/w emulsion, which can be formulated from a water-miscible polymer solvent, such as acetone or ethanol, and the aqueous phase contains a gel, the salting-out agent and a colloidal stabilizer. Examples of suitable salting-out agents include electrolytes, such as magnesium chloride (MgCl₂), calcium chloride (CaCl₂) or magnesium acetate [Mg(CH₃COO)₂], as well as non-electrolytes e.g., sucrose. The miscibility of acetone and water can be reduced by saturating the aqueous phase, which allows the formation of an o/w emulsion from the other miscible phases. The o/w emulsion is prepared, under intense stirring, at room temperature. Subsequently, the emulsion is diluted using an appropriate volume of deionized water or an aqueous solution in order to allow the diffusion of the organic solvent to the external phase, the precipitation of the polymer, and consequently, the formation of nanospheres. The remaining solvent and salting-out agent are eliminated by cross-flow filtration. The condition of complete miscibility between the organic solvent and water is not essential but simplifies the execution process. The dimensions of the nanounits obtained by this method can be about 170 nm to about 900 nm. The average size can be adjusted to values of about 200 nm and 500 nm, by varying polymer concentration of the internal phase/volume of the external phase.

In some embodiments, the method of preparation of a pharmaceutical composition according to embodiments herein may include a wet granulation process including premixing one or more active ingredient and excipients including a binder in a mixer to obtain a pre-mixture; granulating the pre-mixture of (1) by adding the granulation liquid, preferably purified water; drying the granules of (2) in a fluidized bed dryer or a drying oven; optionally dry sieving of the dried granules of (3); mixing the dried granules of (4) with the remaining excipients like glidant and lubricant in a mixer to obtain the final mixture; tableting the final mixture of (5) by compressing it on a suitable tablet press to produce tablets cores; (7) optionally film-coating of the tablet cores of (6) with a non-functional coat.

In some embodiments, the method of preparation of a pharmaceutical composition according to embodiments herein may include a direct compression process for making a pharmaceutical composition, wherein said process comprises the steps of: (1) premixing one or more active ingredient and the main portion of the excipients in a mixer to obtain a pre-mixture; (2) optionally dry screening the pre-mixture through a sereen in order to segregate cohesive particles and to improve content uniformity; (3) mixing the pre-mixture of (1) or (2) in a mixer, optionally by adding remaining excipients to the mixture and continuing mixing; (4) tableting the final mixture of (3) by compressing it on a suitable tablet press to produce the tablet cores; (5) optionally film-coating of the tablet cores of (4) with a non-functional coat.

In some embodiments, the method of preparation of a pharmaceutical composition according to embodiments herein may include a dry granulation process for making a pharmaceutical composition, wherein said process comprises the steps of: (1) mixing one or more active ingredient with either all or a portion of the excipients in a mixer; (2) compacting the mixture of (1) on a suitable roller compactor; (3) reducing the ribbons obtained during (2) to granules, preferably small granules, by suitable milling or sieving steps; (4) optionally mixing the granules of (3) with the remaining excipients in a mixer to obtain the final mixture; (5) tabletting the granules of (3) or the final mixture of (4) by compressing it on a suitable tablet press to produce the tablet cores; (6) optionally film-coating of the tablet cores of (5) with a non-functional coat.

In one or more embodiments, further described herein are methods of preparing a pharmaceutical composition according to embodiments herein. The methods of preparation can include dispersing the cannabinoid and/or the modulator in a liquid (e.g., an oil, a solvent, an antioxidant) and subsequently encapsulating the resulting solution in a hard shell capsule or a soft shell capsule.

In some embodiments, the pharmaceutical compositions as described herein may include a first group of nanounits comprising the cannabinoid and a second group of nanounits comprising the modulator compound. Both groups of nanounits may be mixed together and/or suspend together within an emulsion. In some embodiments, the first group of nanounits may be comprised in a first layer (or core or outer layer) of a solid dosage form and the second group of nanounits may be comprised in a second layer (or core or outer layer) of the solid dosage form, with or without a barrier layer in-between. In one or more embodiments, the first group of nanounits may be encapsulated in a first part of a capsule shell and the second group of nanounits may be encapsulated in a second part of the capsule shell. In at least one embodiment, the first group of nanounits comprising the cannabinoid may be dissolved in a solvent (e.g., an antioxidant oil) and contained in a first portion of a hard or soft shell capsule and the second group of nanounits may be in the form of a dry powder and contained in a second portion of a hard or soft shell capsule.

### Methods of Use

Further described herein are methods of using pharmaceutical compositions according to one or more embodiments herein. In some embodiments, the methods include treating an inflammatory, neurologic and/or vascular condition with one or more embodiments of a pharmaceutical composition as described herein. Inflammatory, neurologic and/or vascular conditions treatable by pharmaceutical compositions according to embodiments herein may include, but are not limited to, neurologic injury or neurologic disease with acute or chronic sequelae that is physical and/or psychological in nature including, but not limited to, insult to neurological tissue (i.e., central nervous system or peripheral nervous system), for example, caused by blunt force trauma (e.g., a concussion), chemical insult to neurological tissue (e.g., chemotherapy or exposure to a chemical agent), an autoimmune response triggered by internal or external stimuli resulting in an inflammatory cascade coupled to activation of NLRP3-inflammasome complex or may be physiologically mediated (e.g., an immune response to an infectious insult or an autoimmune response to various triggers), an encephalopathy, an acute pain condition, a chronic pain condition and/or a combination of any two or more of the foregoing, which can be treated, mitigated or prevented by a timely administration to a subject of an effective amount of a pharmaceutical composition as described herein. The condition may affect neurological tissue, the central nervous system, the peripheral nervous system, the brain, organs, musculature, joints, the pulmonary system, hematologic system, cardiovascular system, neuropsychiatric system, renal system, endocrine system and/or gastrointestinal and hepatobiliary system.

In certain embodiments, the method of treatment includes administering to a patient in need thereof a pharmaceutical formulation as described herein to treat at least one of anxiety, post-traumatic stress syndrome (PTSD), depression, sleep disturbance, myalgic encephalomyelitis/chronic fatigue syndrome (MS/CFS), mononucleosis from Epstein-Barr virus, Lyme Disease, severe acute respiratory syndrome (SARS), COVID-19, post-COVID-19 syndrome, human immunodeficiency virus (HIV), hepatitis B (HBV), hepatitis C (HCV), herpesviridae, Ebola, fibromyalgia, autoimmune encephalitis, neuropathic pain, post-operative cognition deficit, Parkinson's disease, multiple sclerosis (MS), Alzheimer's disease, and/or any combination of two or more of the foregoing.

In one or more embodiments, the method of treatment includes administering to a patient in need thereof a pharmaceutical formulation as described herein to treat a vascular condition including, but not limited to, vasculitis, CNS vasculitis, vascular inflammation, vascular diseases involving activation of NLRP3, NLRP1, NLRC4 or AIM2 inflammasomes, vascular disease resulting from pathogen-driven stimuli, tissue injury or metabolic alterations in multiple cell types and different organs of the body, acute or chronic vascular injury, vasculitides diseases and disorders, Sjögren's disease, Kawasaki's disease, polyarteritis nodosa, Churg-Strauss angiitis, Wegner's granulomatosis, temporal arteritis, isolated angiitis of the central nervous system and/or combinations thereof.

In some embodiments, the methods of treatment include administering to a subject at least one cannabinoid according to one or more embodiments described herein. The method may alternatively or further include administering at least one modulator compound according to one or more embodiments described herein, for example, where the modulator acts as one or more of an NMDA receptor modulator, a 5-HT₃ receptor modulator, a nicotinic acetylcholine receptor modulator, a dopamine D2 receptor modulator, or a sigma-1 receptor modulator.

According to one or more embodiments, the methods of treatment can include administering to a subject a pharmaceutical composition according to embodiments described herein to treat post-infectious encephalopathies and/or post-COVID 19 (“long-haul”) syndrome. Subjects suffering from long-haul COVID-19 syndrome suffer from a range of lingering symptoms. Most subjects having long-haul COVID-19 syndrome experience neurocognitive symptoms such as brain fog. impaired concentration, fatigue and headaches. Insomnia and sleep disturbances are also common. Pulmonary symptoms including shortness of breath and chronic cough have been equally as common. Heart palpitations, depression and anxiety also have been prominent.

In some embodiments, a pharmaceutical composition according to embodiments herein is administered for treating post-COVID 19 syndrome including administering a cannabinoid (e.g., CBD) and/or a modulator (e.g., memantine) to a subject in an effective amount to treat the post-COVID 19 and associated symptoms including those set forth above. In certain embodiments, the pharmaceutical composition may be an oral formulation such as a tablet, capsule, gelcap, sublingual dosage form or depot delivery system, for example, containing nanounits or a nanoemulsion of the cannabinoid and/or modulator.

### Kits

Further described herein are kits containing a pharmaceutical composition according to one or more embodiments. In some embodiments, a kit may include a cannabinoid dosage form as described herein, a modulator dosage form as described herein and optionally a supplement dosage form as described herein. The kit may further include instructions for administering the dosage forms in a suitable manner to achieve a desired therapeutic effect for treating an inflammatory, neurologic and/or vascular condition. For example, the instructions may direct a subject to administer the dosage forms concurrently, simultaneously or sequentially according to a dosing schedule. The dosage forms may be independently in the form of oral, parenteral, sublingual, transdermal, topical and/or transgingival preparations.

In some embodiments, a kit may include tablets or capsules, each tablet or capsule comprising a cannabinoid, a modulator and optionally a supplement. The kit may further include instructions for administering the tablet or capsule in a suitable manner to achieve a desired therapeutic effect for treating an inflammatory, neurologic and/or vascular condition. For example, the instructions may direct a subject to administer the tablet or capsule according to a dosing schedule.

### EXAMPLES

### Example 1 (Prophetic)—A Softgel Capsule Containing CBD and Memantine

Soft gel capsules are prepared by forming a suspension of CBD and memantine particles in a fluid (e.g., an oil). A powder formed of CBD nanoparticles and a powder formed of memantine nanoparticles may be dispersed in the fluid. One or more excipient may be added to the fluid containing the drug particles to form a stable solution having the CBD and memantine each individually at a concentration of about 0.1 wt % to about 20 wt %. The resulting capsules may have the composition as shown in Table 1.

TABLE 1
Prophetic Capsule Formulation

| | Concentration |
|---|---|
| | per unit dose |
| Component | (wt %) |
| Cannabidiol | 0.05-2% |
| Memantine | 0.05-2% |
| Hydroxypropyl | 2.0% |
| Methylcellulose | |
| Vitamin E oil | QS to 10 mL |

### Example 2 (Prophetic)—A Tablet Containing CBD and Memantine

Tablets are prepared by having a CBD core coated with a memantine drug layer. The CBD core may be formed of compressed CBD granules. The memantine drug layer may be spray coated over the CBD core. One or more excipient may be within the CBD core and/or the memantine overlayer. The CBD and memantine each individually is at a concentration of about 0.1 wt % to about 20 wt %. The resulting tablets may have the composition as shown in Table 2.

TABLE 2
Prophetic Tablet Formulation

| | Concentration |
|---|---|
| | per unit dose |
| Component | (wt %) |
| Cannabidiol | 0.5-2% |
| Memantine | 0.5-2% |
| Excipients | QS to 100% |

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

It is to be understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

What is claimed is:

1. A method of treating post-infectious encephalopathy comprising administering to a subject i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator.

2. The method of claim 1, wherein the NMDA receptor modulator is an agonist, an antagonist, or a mixed agonist-antagonist at one or more of the NMDA receptor modulator, the 5-HT₃ receptor modulator, the nicotinic acetylcholine receptor modulator, the dopamine D2 receptor modulator, or the sigma-1 receptor modulator.

3. The method of claim 1, wherein the modulator compound is an NMDA receptor antagonist.

4. The method of claim 1, wherein the modulator compound is glutamatergic NMDA receptor antagonist.

5. The method of claim 1, wherein the modulator compound is 5-HT₃ receptor antagonist.

6. The method of claim 1, wherein the modulator compound is a nicotinic acetylcholine receptor antagonist.

7. The method of claim 1, wherein the modulator compound is a dopamine D2 receptor agonist.

8. The method of claim 1, wherein the modulator compound is a sigma-1 receptor agonist.

9. The method of claim 1, wherein the modulator compound is memantine, a prodrug thereof, an analog thereof, a metabolite thereof or a pharmaceutically acceptable salt thereof.

10. A method of treating post COVID-19 syndrome comprising administering to a subject i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator;
wherein the NMDA receptor modulator is an agonist, an antagonist, or a mixed agonist-antagonist at one or more of the NMDA receptor modulator, the 5-HT₃ receptor modulator, the nicotinic acetylcholine receptor modulator, the dopamine D2 receptor modulator, or the sigma-1 receptor modulator;
wherein the modulator compound is an NMDA receptor antagonist;
wherein the modulator compound is glutamatergic NMDA receptor antagonist;
wherein the modulator compound is 5-HT₃ receptor antagonist;
wherein the modulator compound is a nicotinic acetylcholine receptor antagonist;
wherein the modulator compound is a dopamine D2 receptor agonist;
wherein the modulator compound is a sigma-1 receptor agonist;
wherein the modulator compound is memantine, a prodrug thereof, an analog thereof, a metabolite thereof or a pharmaceutically acceptable salt thereof.

11. A pharmaceutical composition comprising i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator.

12. The pharmaceutical composition of claim 11, wherein the NMDA receptor modulator is an agonist, an antagonist, or a mixed agonist-antagonist at one or more of the NMDA receptor modulator, the 5-HT₃ receptor modulator, the nicotinic acetylcholine receptor modulator, the dopamine D2 receptor modulator, or the sigma-1 receptor modulator.

13. The pharmaceutical composition of claim 11, wherein the modulator compound is an NMDA receptor antagonist.

14. The pharmaceutical composition of claim 11, wherein the modulator compound is 5-HT₃ receptor antagonist.

15. The pharmaceutical composition of claim 11, wherein the modulator compound is a nicotinic acetylcholine receptor antagonist.

16. The pharmaceutical composition of claim 11, wherein the modulator compound is a dopamine D2 receptor agonist.

17. The pharmaceutical composition of claim 11, wherein the modulator compound is a sigma-1 receptor agonist.

18. The pharmaceutical composition of claim 11, wherein the modulator compound is memantine, a prodrug thereof, an analog thereof, a metabolite thereof or a pharmaceutically acceptable salt thereof.

19. The pharmaceutical composition of claim 11, wherein the cannabinoid comprises one or more of cannabinol, cannabidiol, cannabigerol, cannabichromene, Δ9-tetrahydrocannabinol, Δ8-tetrahydrocannabinol, 11-hydroxy-tetrahydrocannabinol, 11-hydroxy-Δ9-tetrahydrocannabinol, levonantradol, Δ11-tetrahydrocannabinol, tetrahydrocannabivarin (THCV), dronabinol, amandamide, nabilone, cannabidivarin (CBDV), a cannavinoid-varin compound, a derivative thereof, a prodrug thereof, an analog thereof, a metabolite thereof or a pharmaceutically acceptable salt thereof.

20. The pharmaceutical composition of claim 11, wherein the cannabinoid is cannabidiol hemisuccinate or tetrahydrocannabinol-hemisuccinate.
